(19) United States
(12) Patent Application Publication
(10) Pub. No.: US 2011/0098723 A1
(43) Pub. Date: Apr. 28, 2011

(54) TICK REMOVAL DEVICE

(75) Inventor: James P. Binkoski, Watertown, CT (US)

(73) Assignee: TICK KEY PRODUCTS, LLC, Torrington, CT (US)

(21) Appl. No.: 12/908,025

(22) Filed: Oct. 20, 2010

Related U.S. Application Data
(60) Provisional application No. 61/254,642, filed on Oct. 23, 2009.

(51) Int. Cl. A61B 17/50; B26F 1/24

(52) U.S. Cl. 606/131; 83/30

(57) ABSTRACT

One example embodiment includes a device for use in the removal of a tick from a host. The device includes a sheet of material and a hole in the sheet of material, where the hole is configured to be placed around a tick. The device also includes a narrow projection, where the narrow projection is configured to grasp and hold the tick during removal, and a transition region. The transition region is configured to connect the hole to the narrow projection and self-center the tick within the device as the device is moved from a position where the tick is in the hole to a position where the tick is in the narrow projection.

### CROSS-REFERENCE TO RELATED APPLICATIONS

This application claims the benefit of and priority to U.S. Provisional Patent Application Ser. No. 61/254,642 filed on Oct. 23, 2009, which application is incorporated herein by reference in its entirety.

### BACKGROUND OF THE INVENTION

Ticks and other blood sucking parasites can pose a huge health risk. For example, there are a number of tick borne diseases including Lyme disease, Q fever, Colorado tick fever, tularemia, tick-borne relapsing fever, babesiosis, ehrlichiosis and Tick-borne meningoencephalitis, as well as anaplasmosis in cattle and canine jaundice.

Some species even stalk the host from ground level, emerging from cracks or crevices located in the woods or even inside a home or kennel, where infestations of “seed ticks” (the six-legged stage of newborn ticks) can attack in huge numbers. Weak or elderly dogs, puppies, and cats are particularly endangered and can die from anemia from a sudden influx of seed ticks. Seed ticks also attack horses, cattle, moose, lions and other mammals, causing anemia, various diseases, paralysis and even death.

Ticks are small enough that detection is often difficult. For example, the ticks can burrow under hair or fur where a cursory inspection can miss their presence. Further, ticks can be difficult to remove once they have attached themselves to a host. Simply grabbing the tick and pulling it will often lead to the body being pulled free from the head, which is embedded in the host. The remaining head can continue to dig into the host producing toxins and removing blood from the host.

Therefore, removal often requires a treatment that forces the tick to voluntarily withdraw from the host. For example, a common method of removal involves putting a match or other heat source near the tick. This carries the risk of burning hair, fur or skin near the tick. In addition, once the heat source is removed, the tick will often begin to suck blood from the host again within a few seconds.

Alternatively, there are some chemicals that cause the ticks to withdraw. However, these chemicals can be dangerous to the host. Often they can cause the host to become sick if used improperly or ingested. These chemicals have to be handled carefully and may not be handy when needed. For example, the chemicals may be left home in a medicine cabinet, meaning that when a user is outdoors away from home and finds an attached tick, the chemicals may not be available for treatment purposes.

In addition, many methods of tick removal are slow. That is it can take several seconds before the tick is removed. During this time, the tick becomes stressed and releases toxins into the host. This increases the chances of causing disease or other damage to the host. This contradicts the purpose of removing the tick, which is to minimize the damage to the host done by the tick.

Accordingly, there is a need in the art for a tick removal device that is quick and easy to use. Additionally, there is a need in the art for a tick removal device that does not rely on heat for tick removal. Further, there is a need in the art for a tick removal device that does not rely on chemicals. In addition, there is a need in the art for a tick removal device that does not leave the head of the tick in the host. Moreover, there is a need in the art for a device that removes the tick from the host and takes the tick far enough away that it cannot immediately seek to reattach itself to the host.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a device for use in the removal of a tick from a host. The device includes a sheet of material and a hole in the sheet of material, where the hole is configured to be placed around a tick. The device also includes a narrow projection, where the narrow projection is configured to grasp and hold the tick during removal, and a transition region. The transition region is configured to connect the hole to the narrow projection and self-center the tick within the device as the device is moved from a position where the tick is in the hole to a position where the tick is in the narrow projection.

Another example embodiment includes a device for use in the removal of a tick from a host. The device includes a sheet of metal, where the sheet of metal is substantially planar, and a hole in the sheet of metal, where the hole is in the plane of the sheet of metal and is configured to be placed over a tick of any size. The device also includes a narrow projection. The narrow projection is configured to grasp and hold the tick during removal and includes an angled edge. The angled edge is configured to fit between the skin of a host and the body of the tick and lift the body of the tick from the host during removal. The device also includes a transition region, where the transition region is configured to connect the hole to the narrow projection and self-center the tick within the device as the device is moved from a position where the tick is in the hole to a position where the tick is in the narrow projection.

Another example embodiment includes a method of manufacturing a device for use in the removal of a tick from a host. The method includes providing a sheet of material and providing a hole in the sheet of material, where in the hole is configured to be placed around a tick. The method also includes providing a narrow projection, where the narrow projection is configured to grasp and hold the tick during removal, and providing a transition region. The transition region is configured to connect the hole to the narrow projection and self-center the tick within the device as the device is moved from a position where the tick is in the hole to a position where the tick is in the narrow projection.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A illustrates a top-view of an example of a tick removal device;

FIG. 1B illustrates a side view of the tick removal device;

FIG. 1C illustrates a bottom view of the tick removal device;

FIG. 1D illustrates a perspective view of the tick removal device;

FIG. 2 illustrates an example of cut away view of a narrow projection;

FIG. 3 is a flowchart illustrating a method of manufacturing a tick removal device; and

FIG. 4 is a flowchart illustrating a method of using a tick removal device.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

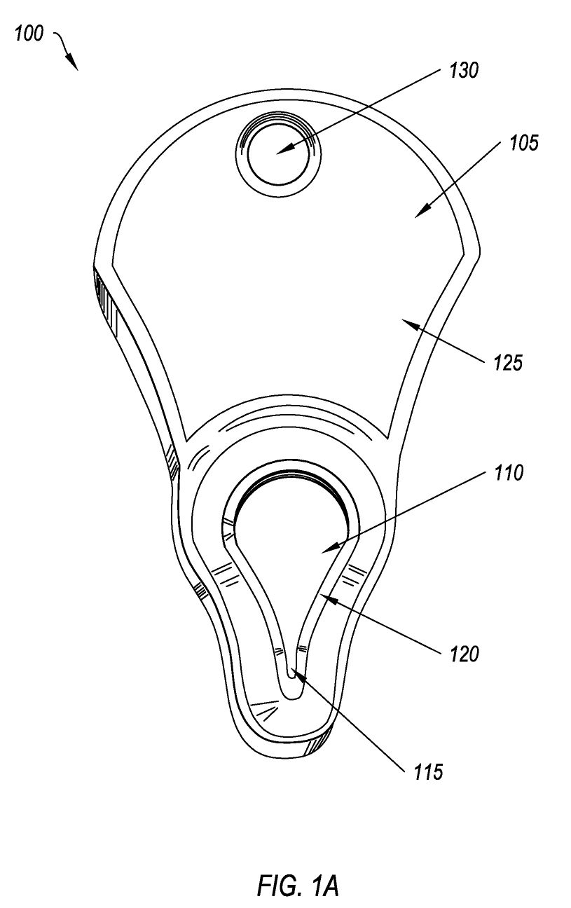

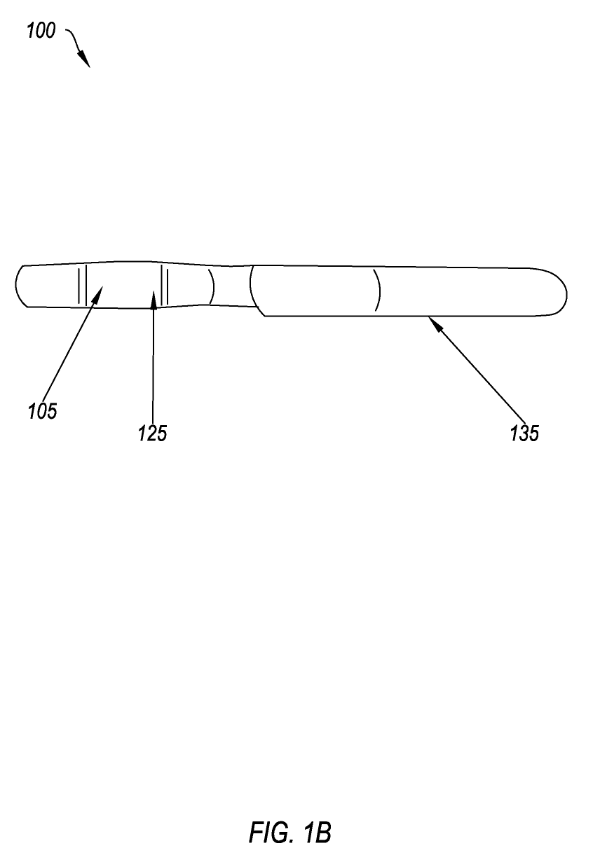

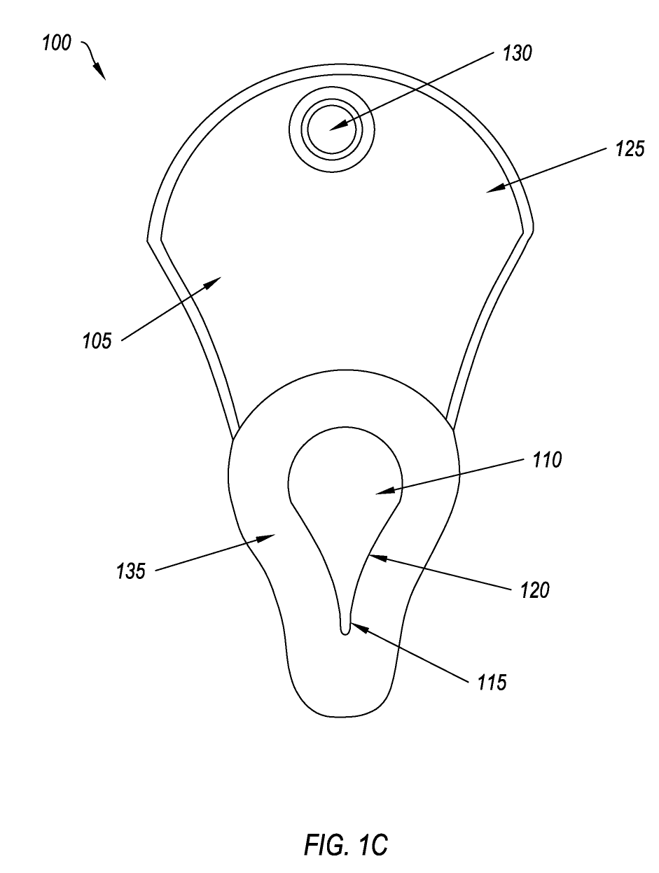

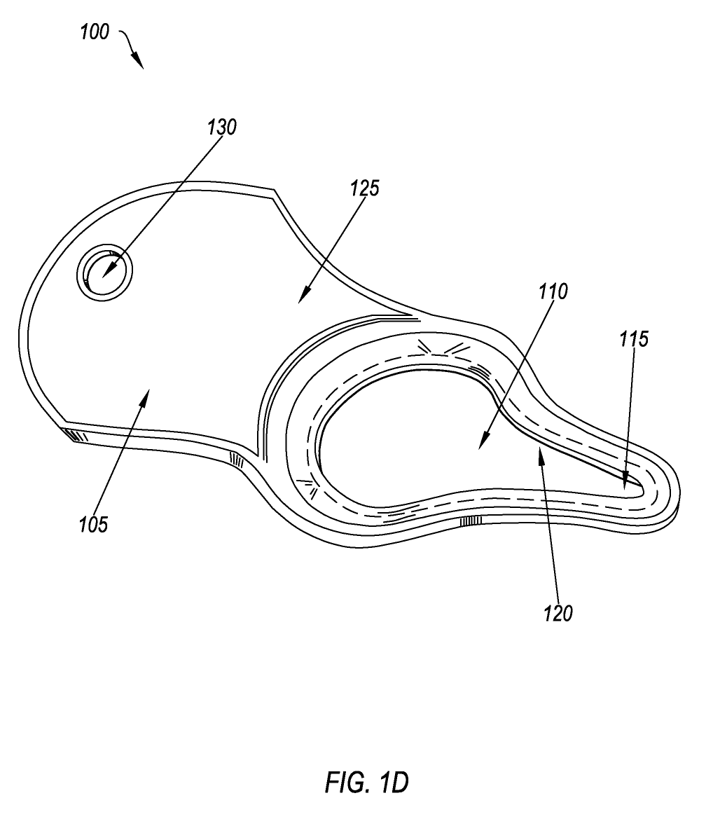

FIGS. 1A, 1B, 1C and 1D illustrate an example of a tick removal device 100. FIG. 1A illustrates a top-view of the tick removal device 100; FIG. 1B illustrates a side view of the tick removal device 100; FIG. 1C illustrates a bottom view of the tick removal device 100; and FIG. 1D illustrates a perspective view of the tick removal device 100.

Ticks are blood-feeding parasites that are often found in tall grass where they will wait to attach to a passing host. A tick will attach itself to its host by inserting its chelicerae (cutting mandibles) and hypostome (feeding tube) into the skin. The hypostome is covered with recurved teeth and serves as an anchor. In at least one implementation, the tick removal device 100 can be used to remove a tick even after the tick has attached itself to a host.

FIGS. 1A, 1B, 1C and 1D show that the tick removal device 100 can include a sheet of material 105. In at least one implementation, the sheet of material 105 can be substantially planar. As used in the specification and the claims, substantially planar shall include any configuration in which the sheet of material 105 includes two surfaces that are substantially parallel to one another with a short edge connecting the two surfaces.

In at least one implementation, the sheet of material 105 can include any material which is suitable for use in a tick removal device. For example, the sheet of material 105 can include metal, such as aluminum or steel. Additionally or alternatively, the sheet of material 105 can include plastic, wood or any other material that is suitable for tick removal. In particular, the sheet of material 105 can include any material which is durable enough to be used in tick removal and can shaped as necessary to be used in tick removal.

In at least one implementation, the sheet of material 105 can be colored. For example, the sheet of material 105 can be made of a colored material or can be painted. Coloring the sheet of material 105 can allow the user to more easily view the tick while the tick removal device 100 is being used. In particular, the color of the tick may show up against the colored sheet of material 105 making it easier for the user to differentiate between the tick removal device 100 and the tick.

FIGS. 1A, 1B, 1C and 1D also show that the tick removal device 100 can include a hole 110 in the material. In at least one implementation, the hole 110 is configured to be placed around a tick of any size. The hole 110 can be between 9.5 and 11.5 millimeters in diameter. For example, the hole 110 can be approximately 10.5 millimeters in diameter. As used in the specification and the claims, the term approximately shall mean that the value is within 10% of the stated value, unless otherwise specified.

In particular, the hole can be large enough to easily be placed around the tick without bumping or otherwise moving the tick. I.e., the hole 110 can allow the tick removal device 100 to be placed around the tick without moving the tick. Moving the tick can cause the tick to inject toxins into the host. In contrast, the hole 110 can allow the tick removal device 100 to be positioned without agitating the tick or otherwise causing the tick to release toxins.

In at least one implementation, the hole 110 passes completely through the sheet of material 105. A hole 110 which passes completely through the sheet of material 105 can allow the user to see the tick while the tick removal device is being used. Allowing the user to see the tick can ensure that the user correctly removes the tick and that the head of the tick does not remain in the host.

FIGS. 1A, 1B, 1C and 1D further show that the tick removal device 100 can include a narrow projection 115. In at least one implementation, the width of the narrow projection 115 can be between 0.25 millimeters and 0.75 millimeters. For example, the width of the narrow projection 115 can be approximately 0.5 millimeters. One of skill in the art will appreciate that the width of the narrow projection 115 can vary over its length. Therefore, as used in the specification and the claims, the term width of the narrow projection 115 shall mean the width between any two points of the narrow projection 115.

In at least one implementation, the narrow projection 115 can allow the tick removal device 100 to grip the tick as it is removed from the host. In particular, as the tick removal device 100 is moved from a position where the tick is in the hole 105 to a position where the tick is in the narrow projection 115, the sides of the narrow projection both grip the tick and pull it from the host. The narrow projection 115 can be configured to lift the tick during removal, as discussed below.

FIGS. 1A, 1B, 1C and 1D also show that the tick removal device 100 can include a transition region 120. In at least one implementation, the transition region 120 can connect the hole 110 to the narrow projection 115. The transition region 120 can be a curved edge. In particular, the transition region 120 can be a curve with a radius between 18 millimeters and 23 millimeters. For example, the transition region 120 can be a curve with a radius of approximately 20.5 millimeters.

A curved edge can allow the tick removal device 100 to self-center as the tick removal device 100 is used to remove a tick. That is, as the tick removal device 100 is moved such that the tick is located in the hole 110 to the narrow projection 115, one curved edge of the transition region 120 can press against the tick. This can force the narrow projection 115 to center on the tick, making removal of the tick easier.

FIGS. 1A, 1B, 1C and 1D also show that the tick removal device 100 can include a handle 125. In at least one implementation, the handle 125 can be gripped by a user while using the tick removal device 100. The handle 125 can be located in the sheet of material on the opposite side of the hole 110 relative to the narrow projection 115. Placing the handle 125 opposite the narrow projection 115 can make moving the tick removal device 100 from a position where the tick is in the hole 110 to a position where the tick is in the narrow projection 115 a natural movement. That is, the user's movement of the handle 125 can naturally move the tick removal device 100 in a manner that removes the tick.

FIGS. 1A, 1B, 1C and 1D further show that the tick removal device 100 can include a second hole 130. In at least one implementation, the second hole 130 can be configured to connect the tick removal device 100 to another device. The second hole 130 can be between 2.5 and 6 millimeters in diameter. In particular, the second hole 130 can be approximately 5 millimeters in diameter.

For example, the second hole 130 can be configured to receive a chain or key ring. This can, in turn, allow the tick removal device 100 to be carried where it is easily accessible. For example, the tick removal device 100 can be connected to a pet collar in order to ensure that the tick removal device 100 can be accessed when needed to remove a tick from the pet.

FIGS. 1A, 1B, 1C and 1D also show that the tick removal device 100 can include a bottom surface 135 that is flat or substantially flat. In at least one implementation, a flat bottom surface 135 can allow the tick removal device 100 to be moved along the skin, hair or fur of the host. In particular, a flat bottom surface 135 can allow the tick removal device 100 to slide while removing a tick, as described below.

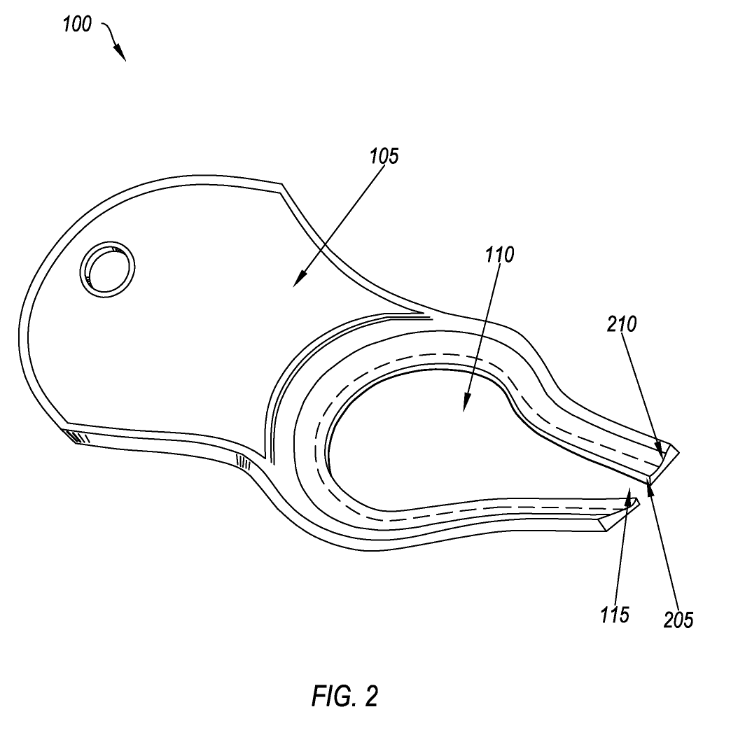

FIG. 2 illustrates an example of cut away view of the narrow projection 115. In at least one implementation, the narrow projection 115 narrows in width as it goes further from the hole 110. This can allow the tick removal device 100 to be used to remove multiple different types and sizes of ticks. In particular, as the tick enters the narrow projection 115, the narrow projection 115 closes in on and removes the tick from the host.

FIG. 2 shows that the narrow projection 115 can include a thin edge 205. In at least one implementation, the thin edge can be between 0.45 millimeters and 0.65 millimeters in thickness. For example, the thin edge can be approximately 0.55 millimeters in thickness. One of skill in the art will appreciate that a thin edge can allow the narrow projection 115 to be used in tick removal without damaging the host or the tick. In particular, if the thin edge 205 is too thin it can cut the skin of the host or cut the head of the tick off during removal. Therefore, the thin edge 205 needs to be thin enough to allow for tick removal but think enough to avoid damaging the host or the tick.

FIG. 2 also shows that the narrow projection 115 can include an angled edge 210. In at least one implementation, the angled edge can transition the narrow projection 115 from the thickness of the thin edge to the thickness of the sheet of material 105. That is, the angled edge 210 strengthens the thin edge 205 by connecting it to the sheet of material 105. The angled edge 210 can be straight or rounded without restriction, unless otherwise specified. The angled edge can be angled between 10 degrees and 25 degrees where it meets the thin edge 205. In particular, the angled edge 210 can be angled at approximately 15 degrees where it meets the thin edge 205.

In at least one implementation, the thin edge 205 and the angled edge 210 are configured to fit between the skin of a host and the body of the tick. That is, the thin edge 205 and the angled edge 210 are thin enough that as the tick removal device 100 moves along the skin, hair or fur of the host the thin edge 205 and the angled edge 210 pass between the body of the tick and the host, allowing the tick removal device to remove the tick, as described below.

In at least one implementation, the thin edge 205 and the angled edge 210 are configured to lift the body of the tick from the host during removal. In particular, as the tick removal device 100 is moved such that the tick is moved along the narrow projection 115 the width of the narrow projection 115 continues to decrease. This, in turn, pushes the thin edge 205 and the angled edge 210 in between the host and the tick. As the tick removal device 100 is then pulled away from the host, the thin edge 205 and the angled edge 210 lift the tick, removing the tick, including the head, from the host. That is, the thin edge 205 and the angled edge 210 provide the proper leverage to remove the tick from the host without disconnecting the tick's head from the tick's body.

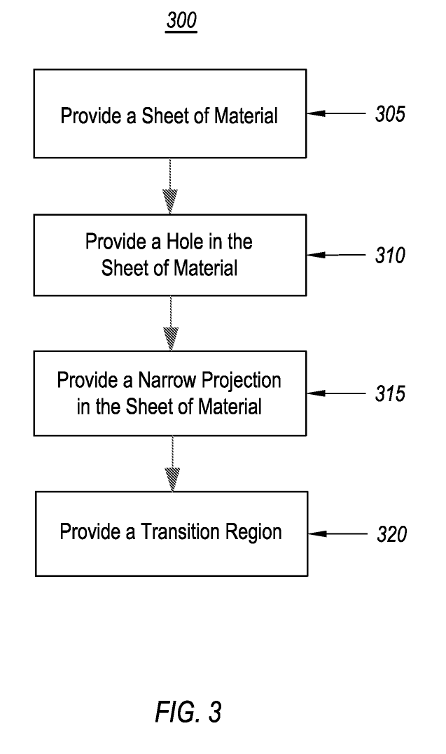

FIG. 3 is a flowchart illustrating a method 300 of manufacturing a tick removal device. One of skill in the art will appreciate that the method 300 can be used to produce the tick removal device 100 of FIG. 1; however, the method 300 can be used to produce a tick removal device other than the tick removal device 100 of FIG. 1.

FIG. 3 show that the method 300 can include providing a sheet of material 305. In at least one implementation, the sheet of material can be substantially planar. That is, the sheet material can include two surfaces that are substantially parallel to one another with a short edge connecting the two surfaces.

In at least one implementation, the sheet of material can include any material which is suitable for use in a tick removal device. For example, the sheet of material can include metal, such as aluminum or steel. Additionally or alternatively, the sheet of material can include plastic, wood or any other material that is suitable for tick removal. In particular, the sheet of material can include any material which is durable enough to be used in tick removal and can shaped as necessary to be used in tick removal.

In at least one implementation, the sheet of material can be colored. For example, the sheet of material can be made of a colored material or can be painted. Coloring the sheet of material can allow the user to more easily view the tick while the tick removal device is being used. In particular, the color of the tick may show up against the colored sheet of material making it easier for the user to differentiate between the tick removal device and the tick.

FIG. 3 also shows that the method 300 can include providing a hole in the sheet of material 310. In at least one implementation, the hole is configured to be placed around a tick of any size. The hole can be between 9.5 and 11.5 millimeters in diameter. For example, the hole can be approximately 10.5 millimeters in diameter.

In particular, the hole can be large enough to easily be placed around the tick without bumping or otherwise moving the tick. I.e., the hole can allow the tick removal device to be placed around the tick without moving the tick. Moving the tick can cause the tick to inject toxins into the host. In contrast, the hole can allow the tick removal device to be positioned without agitating the tick or otherwise causing the tick to release toxins.

In at least one implementation, the hole passes completely through the sheet of material. A hole which passes completely through the sheet of material can allow the user to see the tick while the tick removal device is being used. Allowing the user to see the tick can ensure that the user correctly removes the tick and that the head of the tick does not remain in the host.

FIG. 3 further shows that the method 300 can include providing a narrow projection in the sheet of material 315. In at least one implementation, the width of the narrow projection can be between 0.25 millimeters and 0.75 millimeters. For example, the width of the narrow projection can be approximately 0.5 millimeters.

In at least one implementation, the narrow projection can allow the tick removal device to grip the tick as it is removed from the host. In particular, as the tick removal device is moved from a position where the tick is in the hole to a position where the tick is in the narrow projection, the sides of the narrow projection both grip the tick and pull it from the host. The narrow projection can be configured to lift the tick during removal, as discussed above.

In at least one implementation, the narrow projection can include a thin edge. In at least one implementation, the thin edge can be between 0.45 millimeters and 0.65 millimeters in thickness. For example, the thin edge can be approximately 0.55 millimeters in thickness. One of skill in the art will appreciate that a thin edge can allow the narrow projection to be used in tick removal without damaging the host or the tick. In particular, if the thin edge is too thin it can cut the skin of the host or cut the head of the tick off during removal. Therefore, the thin edge needs to be thin enough to allow for tick removal but think enough to avoid damaging the host or the tick.

In at least one implementation, the narrow projection can include an angled edge. The angled edge can transition the narrow projection from the thickness of the thin edge to the thickness of the sheet of material. That is, the angled edge strengthens the thin edge by connecting it to the sheet of material. The angled edge can be straight or rounded without restriction, unless otherwise specified. The angled edge can be angled between 10 degrees and 25 degrees where it meets the thin edge. In particular, the angled edge can be angled at approximately 15 degrees where it meets the thin edge.

In at least one implementation, the thin edge and the angled edge are configured to fit between the skin of a host and the body of the tick. That is, the thin edge and the angled edge are thin enough that as the tick removal device moves along the skin, hair or fur of the host the thin edge and the angled edge pass between the body of the tick and the host, allowing the tick removal device to remove the tick, as described below.

In at least one implementation, the thin edge and the angled edge are configured to lift the body of the tick from the host during removal. In particular, as the tick removal device is moved such that the tick is moved along the narrow projection the width of the narrow projection continues to decrease. This, in turn, pushes the thin edge and the angled edge in between the host and the tick. As the tick removal device is then pulled away from the host, the thin edge and the angled edge lift the tick, removing the tick, including the head, from the host. That is, the thin edge and the angled edge provide the proper leverage to remove the tick from the host without disconnecting the tick's head from the tick's body.

FIG. 3 also shows that the method can include providing a transition region 320. In at least one implementation, the transition region can connect the hole to the narrow projection. The transition region can be a curved edge. In particular, the transition region can be a curve with a radius between 18 millimeters and 23 millimeters. For example, the transition region can be a curve with a radius of approximately 20.5 millimeters.

A curved edge can allow the tick removal device to self-center as the tick removal device is used to remove a tick. That is, as the tick removal device is moved such that the tick is located in the hole to the narrow projection, one curved edge of the transition region can press against the tick. This can force the narrow projection to center on the tick, making removal of the tick easier.

In at least one implementation, the method 300 can also include providing a handle. The handle can be gripped by a user while using the tick removal device. The handle can be located in the sheet of material on the opposite side of the hole relative to the narrow projection. Placing the handle opposite the narrow projection can make moving the tick removal device from a position where the tick is in the hole to a position where the tick is in the narrow projection a natural movement. That is, the user's movement of the handle can naturally move the tick removal device in a manner that removes the tick.

In at least one implementation, the method can include providing a second hole. The second hole can be configured to connect the tick removal device to another device. The second hole can be between 2.5 and 6 millimeters in diameter. In particular, the second hole can be approximately 5 millimeters in diameter. For example, the second hole can be configured to receive a chain or key ring. This can, in turn, allow the tick removal device to be carried where it is easily accessible. For example, the tick removal device can be connected to a pet collar in order to ensure that the tick removal device can be accessed when needed to remove a tick from the pet.

In at least one implementation, the method 300 can also include providing a bottom surface that is flat or substantially flat. A flat bottom surface can allow the tick removal device to be moved along the skin, hair or fur of the host. In particular, a flat bottom surface can allow the tick removal device to slide while removing a tick, as described above.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

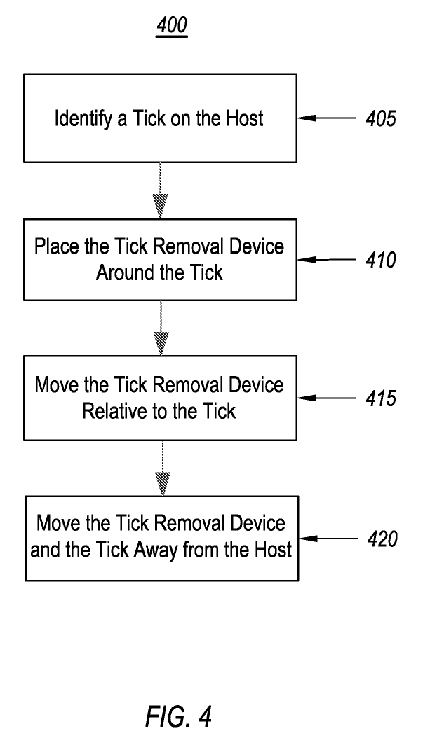

FIG. 4 is a flowchart illustrating a method 400 of using a tick removal device. One of skill in the art will appreciate that the method 400 can be used with the tick removal device 100 of FIG. 1 or a tick removal device manufactured according to the method 300 of FIG. 3; however, the method 400 can be used with a tick removal device other than the tick removal device 100 of FIG. 1 or a tick removal device manufactured according to the method 300 of FIG. 3.

FIG. 4 shows that the method 400 can include identifying a tick on the host 405. In at least one implementation, identifying a tick on the host 405 includes determining whether the tick has begun to feed on the host. Once the tick has begun to feed on the host removal becomes more difficult because the head of the tick is embedded in the host. In particular, removing the tick once the head is embedded in the host is more difficult because the head can be easily removed from the tick's body and left in the host if the tick is removed incorrectly.

FIG. 4 also shows that the method 400 can include placing the tick removal device around the tick 410. In at least one implementation, placing the tick removal device around the tick 410 can include placing the tick removal device such that the tick is within a hole in the tick removal device. In particular, placing the tick removal device around the tick 410 can ensure that the tick removal device and the tick are in proper position relative to one another for the tick to be properly removed.

FIG. 4 further shows that the method 400 can include moving the tick removal device relative to the tick 415. In at least one implementation, moving the tick removal device relative to the tick 415 includes moving the tick removal device such that the tick removal device, or a portion thereof, is in between the host and the tick. In particular, moving the tick removal device relative to the tick 415 can include moving the tick removal device such that the tick moves from a hole in the tick removal device to a narrow projection in the tick removal device.

FIG. 4 also shows that the method 400 includes moving the tick removal device and the tick away from the host 420. In at least one implementation, moving the tick removal device, a portion of which has been placed in between the tick and host, also removes the tick. That is, the tick is automatically removed by moving the tick removal device away from the host.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A device for use in the removal of a tick from a host, the device comprising:
a sheet of material;
a hole in the sheet of material, wherein the hole is configured to be placed around a tick;
a narrow projection, wherein the narrow projection is configured to grasp and hold the tick during removal; and
a transition region, wherein the transition region is configured to:
connect the hole to the narrow projection; and
self-center the tick within the device as the device is moved from a position where the tick is in the hole to a position where the tick is in the narrow projection.

2. The device of claim 1, wherein the sheet of material includes a metal.

3. The device of claim 1, wherein the metal includes one of:
aluminum;
plastic; or
steel.

4. The device of claim 1, wherein the sheet of material is substantially planar.

5. The device of claim 1, wherein the diameter of the hole is between 9.5 millimeters and 11.5 millimeters.

6. The device of claim 5, wherein the diameter of the hole is approximately 10.5 millimeters.

7. The device of claim 1, wherein the width of the narrow projection is between 0.25 millimeters and 0.75 millimeters.

8. The device of claim 7, wherein the width of the narrow projection is approximately 0.5 millimeters.

9. A device for use in the removal of a tick from a host, the device comprising:
a sheet of metal, wherein the sheet of metal is substantially planar;
a hole in the sheet of metal, wherein the hole is in the plane of the sheet of metal and is configured to be placed over a tick of any size;
a narrow projection, wherein the narrow projection:
is configured to grasp and hold the tick during removal; and
includes an angled edge, wherein the angled edge is configured to:
fit between the skin of a host and the body of the tick; and
lift the body of the tick from the host during removal;
a transition region, wherein the transition region is configured to:
connect the hole to the narrow projection; and
self-center the tick within the device as the device is moved from a position where the tick is in the hole to a position where the tick is in the narrow projection.

10. The device of claim 9, wherein the narrow projection includes a thin edge, wherein the thin edge is configured to fit between the tick and the host.

11. The device of claim 10, wherein the thickness of the thin edge is between 0.45 millimeters and 0.65 millimeters.

12. The device of claim 11, wherein the thickness of the thin edge is approximately 0.55 millimeters.

13. A method of manufacturing a device for use in the removal of a tick from a host, the method comprising:
providing a sheet of material;
providing a hole in the sheet of material, wherein the hole is configured to be placed around a tick;
providing a narrow projection, wherein the narrow projection is configured to grasp and hold the tick during removal; and
providing a transition region, wherein the transition region is configured to:
connect the hole to the narrow projection; and
self-center the tick within the device as the device is moved from a position where the tick is in the hole to a position where the tick is in the narrow projection.

14. The device of claim 13 further comprising providing a thin edge in the narrow projection, wherein the thin edge is configured to fit between the tick and the host.

15. The device of claim 14 further comprising providing an angled edge, wherein the angled edge connects the narrow edge to the sheet of material.

16. The device of claim 15, wherein the transition region includes a curve with a radius between 18 millimeters and 23 millimeters.

17. The device of claim 16, wherein the transition region includes a curve with a radius of approximately 20.5 millimeters.

18. The device of claim 13 further comprising a second hole, wherein the second hole is configured to connect to a second device.

19. The device of claim 18, wherein the diameter of the second hole is between 2.5 millimeters and 6 millimeters.

20. The device of claim 19, wherein the diameter of the second hole is approximately 5 millimeters.
